(19) United States
(12) Patent Application Publication
(10) Pub. No.: US 2010/0069325 A1
(43) Pub. Date: Mar. 18, 2010

(54) Pharmaceutical composition for treatment of chronic liver diseases and use thereof

(76) Inventors: Yiyang HU, Shanghai (CN); Ping LIU, Shanghai (CN); Xuemei LI, Shanghai (CN); Qin FENG, Shanghai (CN); Jinghua PENG, Shanghai (CN); Lieming XU, Shanghai (CN); Chenghai LIU, Shanghai (CN)

Correspondence Address: MATTHIAS SCHOLL, 14781 MEMORIAL DRIVE, SUITE 1319, HOUSTON, TX, 77079, US

(21) Appl. No.: 12/558,638

(22) Filed: Sep. 14, 2009

(30) Foreign Application Priority Data
Sep. 12, 2008 (CN) 200810042941.6

(51) Int. Cl. A61K 31/715; A61P 1/16

(52) U.S. Cl. 514/54

(57) ABSTRACT

A pharmaceutical composition for treatment of chronic liver diseases, the composition including at least cordyceps sinensis polysaccharide, amygdaloside, and gypenoside. The pharmaceutical composition slows down liver fibrosis, stops the development thereof, or reverses the same. A method of using the pharmaceutical composition for treatment of chronic liver diseases.

### CROSS-REFERENCE TO RELATED APPLICATIONS

Pursuant to 35 U.S.C. §119 and the Paris Convention Treaty, this application claims priority benefits to Chinese Patent Application No. 200810042941.6 filed on Sep. 12, 2008, the contents of which are incorporated herein by reference.

### BACKGROUND OF THE INVENTION

1. Field of the Invention

The invention relates to a pharmaceutical composition, and more particularly to a pharmaceutical composition for treatment of liver fibrosis and liver injury, as well as its method of use as formulations.

2. Description of the Related Art

Fibrosis is almost a common path in the development of all kinds of chronic liver diseases into liver cirrhosis. The essence of liver fibrosis is excessive accumulation of extracellular matrix (ECM). Slowing, preventing, and reversing liver fibrosis is an important step for the treatment of liver diseases. However, so far, good clinical treatment is still lacked in modern medical field.

Conventional studies on fiber fibrosis focus on the pathogenesis thereof. The pathogenesis of fiber fibrosis is complicated and involves in multiple factors. It is nearly impossible to prevent or treat a complicated disease by merely concentrating on a single step or target. That is to say, for a complicated disease, a comprehensive treatment is needed.

In prior art, some traditional Chinese herbal compositions having functions of anti-liver fibrosis have been disclosed. However, these traditional Chinese herbal compositions have complicated components and proportion thereof, which results in disadvantages such as poor stability and controllability. If treatment is performed by a single ingredient, the significant advantage of treatment of diseases from an overall perspective is lost.

### SUMMARY OF THE INVENTION

Therefore, in view of the above-described problems, it is one objective of the invention to provide a pharmaceutical composition for treatment of chronic liver diseases featuring high capability of anti-liver fibrosis and anti-liver injury.

It is another objective of the invention to provide a method for treatment of chronic liver diseases featuring high capability of anti-liver fibrosis and anti-liver injury.

To achieve the above objectives, in accordance with one embodiment of the invention, provided is a pharmaceutical composition for treatment of chronic liver diseases featuring high capability of anti-liver fibrosis and anti-liver injury, the pharmaceutical composition comprising cordyceps sinensis polysaccharide, amygdaloside, and gypenoside.

In a class of this embodiment, the cordyceps sinensis polysaccharide is extracted from mycelium of cordyceps sinensis.

In a class of this embodiment, the amygdaloside is extracted from peach kernel or almond.

In a class of this embodiment, the gypenoside is extracted from gynostemma.

In a class of this embodiment, a weight ratio of the cordyceps sinensis polysaccharide to amygdaloside to gypenoside is 1:0.2-10:0.2-8, particularly 1:8:2.5, or 1:1.33:0.83.

In a class of this embodiment, the pharmaceutical composition is prepared into a conventional formulation by conventional methods, and a dosage form thereof includes but is not limited to a granule, tablet, and capsule.

In accordance with another embodiment of the invention, provided is a method of treatment of chronic liver diseases featuring high capability of anti-liver fibrosis and anti-liver injury, the method comprising administering to a patient in need thereof a pharmaceutical composition comprising cordyceps sinensis polysaccharide, amygdaloside, and gypenoside.

In a class of this embodiment, the chronic liver disease is chronic hepatitis.

In a class of this embodiment, the chronic liver disease is liver fibrosis.

In a class of this embodiment, the chronic liver disease is liver cirrhosis.

The pharmaceutical composition of the invention was screened and tested by a uniform design method, i.e., administering to animals having DMN- or CCl₄-induced liver injury or liver fibrosis for treatment of liver injury or liver fibrosis by animal model experiments. The results showed, the pharmaceutical composition can significantly reduce the content of collagen fibers of liver tissues of rats, weaken the degree of liver fibrosis and liver injury, and the effect is much better than a single component of the composition. Therefore, the pharmaceutical composition has capability of slowing liver fibrosis, of stopping the development of liver fibrosis, and of prompting the reverse of liver fibrosis. All in all, the pharmaceutical composition has capability of treatment of chronic hepatitis, liver fibrosis, and liver cirrhosis.

### BRIEF DESCRIPTION OF THE DRAWINGS

The invention is described hereinbelow with reference to accompanying drawings, in which:

FIG. 1 is images of collagen stained by Sirius red according to Example 5 (×100), and 1: Normal group; 2: Model group; 3: Cordyceps sinensis polysaccharide group; 4. Gypenoside group; 5: Amygdaloside group; 6: Composition group; 7: Fuzhenghuayu capsule group; 8: Colchicine group; and

FIG. 2 is images of collagen stained by Sirius red according to Example 6 (×100), and 1: Normal group; 2: Model group; 3: Cordyceps sinensis polysaccharide group; 4. Gypenoside group; 5: Amygdaloside group; 6: Composition group; 7: Fuzhenghuayu capsule group; 8: Colchicine group.

### DETAILED DESCRIPTION OF THE EMBODIMENTS

For further illustrating the invention, experiments detailing a pharmaceutical composition for treatment of chronic liver diseases and use thereof are described below. It should be noted that the following examples are intended to describe and not to limit the invention.

### Example 1

### Composition Screening Experiments on Rats Having Dimethyinitrosamine-Induced Liver Fibrosis by Uniform Design Method

Materials and Method

Animals: Wistar male rats of clean grade, weighted 160±10 g, and provided by Shanghai Laboratory Animal Center, Chinese Academy of Sciences.

Reagents and Drugs:
1) Dimethylnitrosamine (DMN), manufactured by Nippon Kasei Chemical Co., Ltd;
2) Cordyceps sinensis polysaccharide, extracted from Cordyceps sinensis and manufactured by Shanghai Kangzhou Fungi Extract Co., Ltd, Specification: 95%; Batch number: 20060828;
3) Salvianolic acid B, extracted from Salvia and manufactured by Shanghai Institute of Materia Medica, Chinese Academy of Sciences, Specification: more than 60%;
4) Amygdaloside, extracted from almond and manufactured by Xi'an Guanyu Bio-tech Co., Ltd., Specification: 98%; Batch number: 06061; and
5) Gypenoside, extracted from gynostemma and manufactured by Xi'an Honson Biotechnology Co., Ltd., Specification: 98%; Batch number: GY060913.

Composition screening experiments by uniform design method: 94 rats were randomly divided into a normal group (5) and modeling groups (89). The modeling groups were administered 0.5% DMN (2 mL/Kg) by intraperitoneal injection for three consecutive days each week. Four weeks later, 5 rats from the modeling groups died. The remaining 84 rats were randomly divided into 8 groups with each 9-10 according to a uniform design method (to be described hereinafter). 9 rats from the modeling groups were selected to verify the validity of the modeling groups. Subsequently, the modeling groups were administered corresponding composition (10 mL/Kg) by gavage according to the uniform design method for 2 weeks, 6 times each week. The normal group was administered normal saline by intraperitoneal injection in the first 4 weeks, and drinking water by gavage in the following two weeks. After two weeks of treatment, the remaining 67 rats were anesthetized by intraperitoneal injection of 2% pentobarbital sodium (2 mL/Kg). Blood was collected from inferior vena cava, and blood serum was separated by centrifugation. Liver tissue was also collected for testing.

Uniform design method: It is well known that Fuzhenghuayu capsule, a traditional Chinese medicine having functions of anti-liver injury and anti-liver fibrosis, mainly consists of four components of Cordyceps sinensis polysaccharide, Salvianolic acid B, Amygdaloside, and Gypenoside. Based on a uniform design form of U⁹ (9⁸), the four therapeutically effective components were selected as research factors. Each factor had eight dose levels, i.e., eight different dose by gradual increase. The eight doses of Cordyceps sinensis polysaccharide (X₁) and Amygdaloside (X₃) was 20-160 mg, Salvianolic acid B (X₂) 4-32 mg, and Gypenoside (X₄) 50-400 mg. Each factor and dose thereof was determined according to the uniform design form, totally there were eight dose groups.

Screening index: the content of hydroxyproline (Hyp) in liver tissues (Jamall's method).

The obtained data was analysed statistically with software SPSS 12.0.

Stepwise regression analysis was used for the uniform design experiment (statistical test was performed with p=0.05 as a significant level).

Results of Composition Screening Experiments

The content of Hyp in liver tissues from modeling groups (492±66 μg/g liver tissue) was much higher than that of the normal group (191±6 μg/g liver tissue) (p<0.01), and the degree of liver fibrosis was heavy, which implied the modeling was successful. Based on that, the content of Hyp in liver tissues was measured and stepwise regression analysis was performed to give a regression equation, Y=0.004 X1X4−0.003 X3X4+441.763, as shown in Table 1. Results of composition screening experiments showed, when the composition comprised the components X1, X3, and X4, and X1=60 mg, X3=80 mg, X4=50 mg, i.e., the weight ratio was 1:1.33:0.83 (named as Composition A), its effect of anti-liver fibrosis on DMN-induced rats exhibited well.

TABLE 1
Stepwise regression analysis of content
of Hyp in liver tissues from rats

| Groups | n | Average (Hyp) | Regression equation |
|---|---|---|---|
| 1 | 7 | 388 | Y = 0.004 X1X4 − 0.003 |
| 2 | 6 | 337 | X3X4 + 441.763 |
| 3 | 7 | 438 | |
| 4 | 7 | 402 | |
| 5 | 7 | 445 | |
| 6 | 6 | 416 | |
| 7 | 7 | 439 | |
| 8 | 8 | 320 | |

### Example 2

Composition Screening Experiments on Rats Having Carbon Tetrachloride-Induced Liver Fibrosis by Uniform Design Method

Animals: Wistar male rats (90) of SPF grade, weighted 170±15 g, and provided by Shanghai Laboratory Animal Center, Chinese Academy of Sciences.

Reagents and Drugs:

Carbon tetrachloride (CCl₄) and olive oil were purchased from Chinese Pharmaceutical Group; and Cordyceps sinensis polysaccharide, Salvianolic acid B, Amygdaloside, and Gypenoside were the same as those in Example 1.

Composition screening experiments by uniform design method: 77 rats were randomly divided into a normal group (7) and modeling groups (70). The modeling groups were administered 100% CCl₄ (3 mL/Kg) by intraperitoneal injection at the first time, and then followed by an olive oil solution having 50% CCl₄ (2 mL/Kg) for 9 weeks, 2 times each week. The normal group was administered the same dose of olive oil. 6 weeks later, 3 rats from the modeling groups died. The remaining 67 rats were randomly divided into 8 groups with each 7-8 according to a uniform design method. 8 rats from the modeling groups were selected to verify the validity of the modeling groups. After 3 weeks of treatment while modeling, the remaining rats were anesthetized by intraperitoneal injection of 2% pentobarbital sodium. Blood was collected from inferior vena cava, and blood serum was separated by centrifugation. Liver tissue was also collected in centrifuge tubes (1.5 mL) for analyzing.

Uniform design method: following the method disclosed in Example 1.

Screening index: the content of hydroxyproline (Hyp) in liver tissues (Jamall's method).

The obtained data was analysed statistically with software SPSS 12.0. Stepwise regression analysis was used for the uniform design experiment (statistical test was performed with p=0.05 as a significant level).

Results of Composition Screening Experiments

The content of Hyp in liver tissues from modeling groups (442±51 μg/g liver tissue) was much higher than that of the normal group (175±22 μg/g liver tissue) (p<0.01), which implied the modeling was successful. Based on that, the content of Hyp in liver tissues was measured and stepwise regression analysis was performed to give a regression equation, Y=0.006 X1X4−0.633 X3+360.832, as shown in Table 2. Results of composition screening experiments showed, when the composition comprised the components X1, X3, and X4, and X1=20 mg, X3=160 mg, X4=50 mg, i.e., the weight ratio was 1:8:2.5 (named as Composition B), its effect of anti-liver fibrosis on CCl₄-induced rats exhibited well.

TABLE 2
Stepwise regression analysis of content
of Hyp in liver tissues from rats

| Groups | n | Average (Hyp) | Regression equation |
|---|---|---|---|
| 1 | 8 | 378 ± 30 | Y = 0.006 X1X4 − 0.633 |
| 2 | 8 | 357 ± 55 | X3 + 360.832 |
| 3 | 7 | 487 ± 99 | |
| 4 | 7 | 424 ± 74 | |
| 5 | 7 | 487 ± 75 | |
| 6 | 7 | 439 ± 54 | |
| 7 | 7 | 504 ± 62 | |
| 8 | 8 | 457 ± 61 | |

### Example 3

### Therapeutical Effect Testing of Composition A and B on Rats Having DMN-Induced Liver Fibrosis

Materials and Method

Animals: SD male rats of clean grade, weighted 170±10 g, and provided by Shanghai Laboratory Animal Center, Chinese Academy of Sciences.

Reagents and Drugs:
1) Raw materials of Fuzhenghuayu capsule (a traditional Chinese medicine having functions of anti-liver injury and anti-liver fibrosis), manufactured by Shanghai Modern Chinese Medicine Technology Development Co., Ltd.;
2) Kits for liver function test, purchased from Jiancheng Bio-engineering Research Institute of Nanjing; and
3) other materials: the same as those in Example 1.

Grouping and modeling: 60 SD rats were randomly divided into a normal group (5) and modeling groups (55). The modeling method was the same as that in Example 1. Four weeks later, 6 rats from the modeling group died. The remaining 49 rats were randomly divided into a model group (9), a Composition A group (8), a Composition B group (8), a group of Composition A+Salvianolic acid B (8), a group of Composition B+Salvianolic acid B (8) (the added amount of Salvianolic acid B was 12.5 mg/Kg), a control group of Fuzhenghuayu capsule (8). The administered dose of Fuzhenghuayu capsule was 4.6 g/Kg, and the model group was administered the same dose of drinking water. After two weeks of treatment, the remaining rats were anesthetized by intraperitoneal injection of 2% pentobarbital sodium. Blood was collected from inferior vena cava. Liver tissue was also collected for testing.

Screening index: the content of hydroxyproline (Hyp) in liver tissues (Jamall's method), the activity of ALT, AST, and GGT of blood serum, and the content of Alb and Tbil of blood serum.

The obtained data was expressed as x̅±s and analysed statistically with software SPSS 12.0 for variance analysis and comparison between groups.

Results: Compared with that of the normal group, the content of Hyp in liver tissues, the activity of ALT, AST, and GGT, and the content of Tbil of blood serum from the model group increased significantly, while the content of Alb decreased significantly. The content of Hyp in liver tissues, ALT, AST, GGT, and Tbil of blood serum from the Composition A group was much lower than that of the model group, while the content of Alb was much higher than that of the model group, and there was no statistical difference in therapeutical effect between the Composition A group and the control group of Fuzhenghuayu capsule. The index of the Composition B group was similar to that of the Composition A group except that the GGT activity did not decreased. However, after Salvianolic acid B added, the therapeutical effect of both the Composition A group and the Composition B group decreased.

Conclusion: the Composition A and the Composition B screened by a uniform design method had significant therapeutical effect on rats having DMN-induced liver fibrosis and liver injury. Additionally, that the addition of Salvianolic acid B decreased the therapeutical effect of the Compositions A and B proved the therapeutical effect of the composition can be strengthened or weakened.

TABLE 3
Content of Hyp in liver tissues, activity of ALT and AST
of blood serum of rats ( x̅ ± s)

| | | Hyp (μg · g⁻¹ | | |
|---|---|---|---|---|
| Groups | Number | liver weight) | ALT(U · ml⁻¹) | AST(U · ml⁻¹) |
| Normal | 5 | 180 ± 18 ** | 37 ± 7 ** | 17 ± 6 ** |
| Model | 6 | 432 ± 73 | 140 ± 41 | 79 ± 21 |
| Composition A | 6 | 326 ± 21 ** | 84 ± 18 * | 44 ± 9 ** |
| Composition B | 6 | 347 ± 51 * | 93 ± 20 * | 41 ± 14 ** |
| Composition A + | 6 | 348 ± 35 * | 107 ± 20 Δ | 57 ± 10 * Δ |
| Salvianolic acid B | | | | |
| Composition B + | 7 | 385 ± 77 | 135 ± 38 Δ | 65 ± 15 Δ |
| Salvianolic acid B | | | | |
| Fuzhenghuayu | 7 | 294 ± 49 ** | 86 ± 20 ** | 40 ± 14 ** |

TABLE 4
Activity of GGT and content of Tbil and Alb of blood serum of rats ( x̅ ± s)

| Groups | Number | GGT(U · ml⁻¹) | Tbil (mg · dl⁻¹) | Alb(g · l⁻¹) |
|---|---|---|---|---|
| Normal | 5 | 9.75 ± 4.32 ** | 0.39 ± 0.08 ** | 34.08 ± 1.63 ** |
| Model | 6 | 55.63 ± 14.21 | 1.17 ± 0.28 | 24.22 ± 1.33 |
| Composition A | 6 | 28.54 ± 10.85 ** | 0.65 ± 0.20 ** | 30.68 ± 2.27 ** |
| Composition B | 6 | 39.38 ± 17.58 # | 0.67 ± 0.37 * | 29.96 ± 2.38 ** |
| Composition A + | 6 | 37.19 ± 8.64 * | 0.76 ± 0.19 * | 28.65 ± 3.40 ** |
| Salvianolic acid B | | | | |
| Composition B + | 7 | 42.84 ± 12.19 | 0.83 ± 1.34 | 27.53 ± 4.43 * |
| Salvianolic acid B | | | | |
| Fuzhenghuayu | 7 | 22.50 ± 12.91 ** | 0.62 ± 0.11 ** | 30.53 ± 2.33 ** |

Note:
Compared with the model group,
* p < 0.05,
** p < 0.01;
Compared with the composition group,
Δ p < 0.05.

### Example 4

### Therapeutical Effect Testing of Composition A and B on Rats Having CCl₄-Induced Liver Fibrosis

Materials and Method

Animals: SD male rats of SPF grade, weighted 170±10 g, and provided by Shanghai Laboratory Animal Center, Chinese Academy of Sciences.

Reagents and Drugs:
1) Raw materials of Fuzhenghuayu capsule (a traditional Chinese medicine having functions of anti-liver injury and anti-liver fibrosis), manufactured by Shanghai Modern Chinese Medicine Technology Development Co., Ltd.; and
2) other materials: the same as those in Example 2.

Grouping and modeling: 55 SD rats were randomly divided into a normal group (5) and modeling groups (50). The modeling method was the same as that in Example 2. Six weeks later, 2 rats from the modeling group died. The remaining 48 rats were randomly divided into a model group, a Composition A group, a Composition B group, a group of Composition A+Salvianolic acid B, a group of Composition B+Salvianolic acid B, and a control group of Fuzhenghuayu capsule. The administered dose of each group was the same as that in Example 3. After 3 weeks of treatment, the remaining rats were anesthetized by intraperitoneal injection of 2% pentobarbital sodium. Blood was collected from inferior vena cava. Liver tissue was also collected for testing.

Screening index: the content of hydroxyproline (Hyp) in liver tissues (Jamall's method), the activity of ALT, AST, and GGT of blood serum, and the content of Alb and Tbil of blood serum.

The obtained data was expressed as x̅±s and analysed statistically with software SPSS 12.0 for variance analysis and comparison between groups.

Results: Compared with that of the normal group, the content of Hyp in liver tissues, the activity of ALT, AST, and GGT, and the content of Tbil of blood serum from the model group increased significantly, while the content of Alb decreased significantly. The content of Hyp in liver tissues, ALT, AST, GGT, and Tbil of blood serum from the Composition A group and the Composition B group was much lower than that of the model group, while the content of Alb was much higher than that of the model group, and there was no statistical difference in therapeutical effect between the Composition groups and the control group of Fuzhenghuayu capsule. However, after Salvianolic acid B added, the therapeutical effect of both the Composition A group and the Composition B group decreased (Hyp, Tbil of the Composition A group exhibited no significant difference compared with that of the model group, GGT of the Composition B group exhibited no significant difference compared with that of the model group).

Conclusion: the Composition A and the Composition B screened by a uniform design method had significant therapeutical effect on rats having CCl₄-induced liver fibrosis and liver injury. Additionally, that the addition of Salvianolic acid B decreased the therapeutical effect of the Compositions A and B proved the therapeutical effect of the composition can be strengthened or weakened, which further proved the conclusion of Example 3.

TABLE 5
Content of Hyp in liver tissues, activity of ALT and AST
of blood serum of rats ( x̅ ± s)

| | | Hyp (μg · g⁻¹ | | |
|---|---|---|---|---|
| Groups | Number | liver weight) | ALT(U · ml⁻¹) | AST(U · ml⁻¹) |
| Normal | 5 | 134.35 ± 34.75** | 31.65 ± 10.19** | 48.56 ± 8.65** |
| Model | 7 | 384.62 ± 54.56 | 161.23 ± 28.38 | 142.70 ± 27.76 |
| Composition A | 7 | 241.82 ± 40.93** | 86.86 ± 29.00** | 89.02 ± 12.79** |
| Composition B | 6 | 250.35 ± 72.97** | 92.91 ± 31.26** | 85.73 ± 20.58** |
| Composition A + | 7 | 304.77 ± 84.17 | 115.30 ± 32.12* | 112.83 ± 36.15* |
| Salvianolic acid B | | | | |
| Composition B + | 7 | 265.84 ± 100.45* | 122.69 ± 44.58 | 97.18 ± 21.28** |
| Salvianolic acid B | | | | |
| Fuzhenghuayu | 7 | 230.77 ± 91.92** | 80.10 ± 28.87** | 88.46 ± 20.55** |

Note:
Compared with the model group,
*p < 0.05,
**p < 0.01.

TABLE 6
Activity of GGT and content of Tbil and Alb of blood serum of rats ( x̅ ± s)

| Groups | Number | GGT(U · ml⁻¹) | Tbil (mg · dl⁻¹) | Alb(g · l⁻¹) |
|---|---|---|---|---|
| Normal | 5 | 11.86 ± 2.75** | 0.49 ± 0.14** | 35.95 ± 1.50** |
| Model | 7 | 53.78 ± 12.97 | 1.35 ± 0.31 | 27.98 ± 2.21 |
| Composition A | 7 | 31.55 ± 14.56* | 0.91 ± 0.23* | 31.64 ± 3.51* |
| Composition B | 6 | 34.67 ± 7.19* | 0.82 ± 0.32* | 31.17 ± 1.85* |
| Composition A + | 7 | 45.42 ± 13.45* | 1.02 ± 0.58 | 30.26 ± 1.15* |
| Salvianolic acid B | | | | |
| Composition B + | 7 | 40.80 ± 13.62 | 0.91 ± 0.31* | 29.83 ± 1.68* |
| Salvianolic acid B | | | | |
| Fuzhenghuayu | 7 | 28.78 ± 10.06** | 0.79 ± 0.15** | 32.59 ± 2.61** |

Note:
Compared with the model group,
*p < 0.05,
**p < 0.01.

### Example 5

### Comparison Experiments of Therapeutic Effect Between Composition and Single Component on Rats Having DMN-Induced Liver Fibrosis

Materials and Method

Colchicine, manufactured by Yunnan Xishuangbanna Pharmaceutical Co., Ltd., Batch number: 070705; other materials were the same as those in Example 3.

Modeling and grouping: 80 SD rats were randomly divided into a normal group (6) and modeling groups (74). The modeling method was the same as that in Example 1. Four weeks later, 4 rats from the modeling group died. The remaining 76 rats were randomly divided into a model group, a Composition A group, groups having a single component of Composition A, a first control group of Fuzhenghuayu capsule, and a second control group of colchicines, each group 10 rats. The administered dose of colchicines was 0.1 mg/Kg, Fuzhenghuayu capsule 4.6 g/Kg. The Composition A group was administered Cordyceps sinensis polysaccharide 60 mg/Kg, Gypenoside 50 mg/Kg, and Amygdaloside 80 mg/Kg. The administered dose of the Gypenoside group was 200 mg/Kg, Amygdaloside 80 mg/Kg, and Cordyceps sinensis polysaccharide 60 mg/Kg. Each group was administered by gavage in a volume of 10 mL/Kg. The model group was administered the same volume of drinking water. After two weeks of treatment, the remaining 61 rats were anesthetized by intraperitoneal injection of 2% pentobarbital sodium. Blood was collected from inferior vena cava, and blood serum was separated by centrifugation. Liver tissue was also collected in centrifuge tubes (1.5 mL) for analyzing. Two liver tissues from a hepatic lobule was collected and immersed in a formalin solution for testing.

Observing index and method: the content of hydroxyproline (Hyp) in liver tissues (Jamall's method); liver function of blood serum; liver tissues were stained with HE and Sirius red respectively to determine the degree of liver fibrosis according to the S0-S4 staging criteria in literatures.

The obtained data was expressed as x̅±s and analysed statistically with software SPSS 12.0 for variance analysis and comparison between groups. The comparison of ranked data between groups was performed by Ridit analysis.

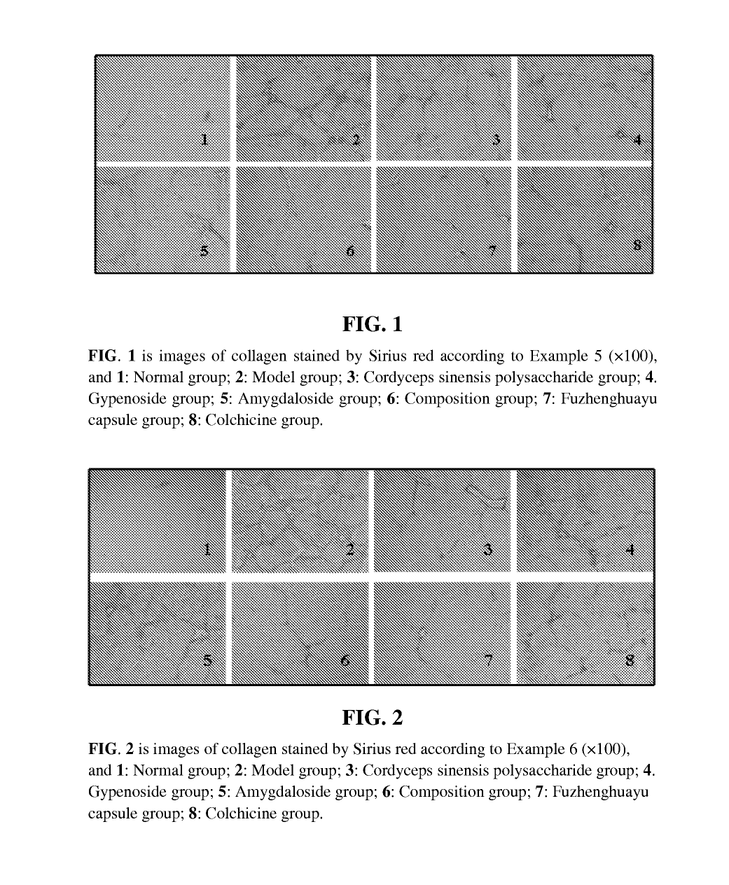

Results: Each index in liver tissues from the model group had significantly increased compared with that of the normal group. The single component of Cordyceps sinensis polysaccharide, Gypenoside, or Amygdaloside can weaken the DMN-induced liver fibrosis, but the Composition A was more effective than the three single component or colchicine (P<0.05). The Sirius red staining of liver tissues showed, in the normal group, the structure of hepatic lobule was normal, only a few of collagen fibers occurred, and no fibroplasias; in the model group, the structure of hepatic lobule was abnormal, a large amount of collagen fibers occurred, and the collagen fibers were broadband-like and connected with each other; in the colchicine group, collagen fibers was decreased as compared with the model group (P<0.05); in the group of Fuzhenghuayu capsule and the Composition group, collagen fibers was significantly decreased as compared with the model group (P<0.01), and the hepatic lobule was basically back to normal, as showed in FIG. 1. Additionally, the Composition of the invention had significant effect against liver injury, and the effect was much better than that of single component or colchicine.

TABLE 7
Content of Hyp in liver tissues, activity of ALT and AST
of blood serum of rats ( x̅ ± s)

| | | Hyp(μg · g⁻¹ | | |
|---|---|---|---|---|
| Groups | N | liver weight) | ALT(U · ml⁻¹) | AST(U · ml⁻¹) |
| Normal | 6 | 165 ± 35** | 17.69 ± 2.95** | 38.14 ± 3.14** |
| Model | 8 | 435 ± 91 | 75.50 ± 11.29 | 107.36 ± 13.69 |
| Cordyceps sinensis | 7 | 338 ± 70*# | 61.15 ± 18.91# | 77.31 ± 15.09* |
| polysaccharide | | | | |
| Gypenosides | 8 | 318 ± 54*# | 43.94 ± 17.62** | 68.02 ± 16.15** |
| Amygdaloside | 8 | 323 ± 79*# | 55.39 ± 15.56*# | 69.13 ± 15.74* |
| Composition A | 8 | 254 ± 39** | 38.43 ± 14.70** | 58.65 ± 25.95** |
| Fuzhenghuayu | 7 | 259 ± 58** | 28.75 ± 6.77** | 54.00 ± 6.44** |
| capsule | | | | |
| Colchicine | 8 | 300 ± 47**# | 47.55 ± 17.47** | 62.81 ± 13.04** |

Note:
Compared with the model group,
*p < 0.05,
**p < 0.01; Compared with the composition A group,
#p < 0.05,
##p < 0.01.

TABLE 8
Activity of GGT and content of Tbil of blood serum of rats ( x̅ ± s)

| Groups | N | GGT(U · ml⁻¹) | Tbil (mg · dl⁻¹) |
|---|---|---|---|
| Normal | 6 | 10.62 ± 2.99** | 0.46 ± 0.14** |
| Model | 8 | 49.40 ± 19.46 | 1.58 ± 0.70 |
| Cordyceps sinensis | 7 | 39.19 ± 16.92## | 0.79 ± 0.30*## |
| polysaccharide | | | |
| Gypenosides | 8 | 26.09 ± 6.10**## | 0.64 ± 0.22**# |
| Amygdaloside | 8 | 37.01 ± 19.92## | 0.95 ± 0.58# |
| Composition A | 8 | 18.05 ± 7.67** | 0.38 ± 0.21** |
| Fuzhenghuayu | 7 | 15.55 ± 4.96** | 0.51 ± 0.51** |
| capsule | | | |
| Colchicine | 8 | 31.30 ± 17.21*## | 0.86 ± 0.56* |

Note:
Compared with the model group,
*p < 0.05,
**p < 0.01;
Compared with the composition A group,
#p < 0.05,
##p < 0.01.

TABLE 9
Comparison of liver fibrosis degree of rats between groups

| | | Degree of liver fibrosis | | | | |
|---|---|---|---|---|---|---|
| Groups | N | S0 | S1 | S2 | S3 | S4 |
| Normal ** | 6 | 6 | 0 | 0 | 0 | |
| Model | 8 | 0 | 0 | 3 | 4 | 1 |
| Cordyceps sinensis | 7 | 0 | 0 | 5 | 2 | 0 |
| polysaccharide | | | | | | |
| Gypenosides | 8 | 0 | 0 | 6 | 2 | 0 |
| Amygdaloside | 8 | 0 | 1 | 4 | 3 | 0 |
| Composition A ** | 8 | 0 | 1 | 7 | 0 | 0 |
| Fuzhenghuayu | 8 | 0 | 2 | 6 | 0 | 0 |
| capsule ** | | | | | | |
| Colchicine * | 8 | 0 | 1 | 5 | 2 | 0 |

Note:
Compared with the model group,
* p < 0.05,
** p < 0.01.

Conclusion: the composition of the invention had significant therapeutical effect on rats having DMN-induced liver fibrosis and injury, and the effect was better than a single component.

### Example 6

### Comparison Experiments of Therapeutic Effect Between Composition and Single Component on Rats Having CCl₄-Induced Liver Fibrosis

Materials and Method

Colchicine, manufactured by Yunnan Xishuangbanna Pharmaceutical Co., Ltd., Batch number: 070705; other materials were the same as those in Example 4.

Modeling and grouping: 86 SD rats were randomly divided into a normal group (6) and modeling groups (80). The modeling method was the same as that in Example 2. Six weeks later, 2 rats from the modeling group died. The remaining 78 rats were randomly divided into a model group (12), a Composition A group (11), groups having a single component of Composition A (each group with 11 rats), a first control group of Fuzhenghuayu capsule (11), and a second control group of colchicine (11). The administered dose was the same as those in Example 5. After three weeks of treatment while modeling, the remaining 81 rats were anesthetized by intraperitoneal injection of 2% pentobarbital sodium. Blood was collected from inferior vena cava, and blood serum was separated by centrifugation. Liver tissue was also collected in centrifuge tubes (1.5 mL) for analyzing. Two liver tissues from a hepatic lobule was collected and immersed in a formalin solution for testing.

Observing index and method: the same as those in Example 5.

The obtained data was expressed as x̅±s and analysed statistically with software SPSS 12.0 for variance analysis and comparison between groups.

Results: Each index in liver tissues from the model group had significantly increased compared with that of the normal group. The Composition A was significantly more effective than the single component of Cordyceps sinensis polysaccharide, Gypenosides, Amygdaloside, or colchicines in weakening the CCl₄-induced liver fibrosis. The Sirius red staining of liver tissues showed, in the normal group, the structure of hepatic lobule was normal, only a few of collagen fibers occurred, and no fibroplasias; in the model group, the structure of hepatic lobule was abnormal, a large amount of collagen fibers occurred, and the collagen fibers were broadband-like and connected with each other; in the colchicine group, collagen fibers was decreased as compared with the model group; in the group of Fuzhenghuayu capsule and the Composition group, collagen fibers was significantly decreased as compared with the model group, and the hepatic lobule was basically back to normal, as showed in FIG. 2.

TABLE 10
Content of Hyp in liver tissues, activity of ALT and AST
of blood serum of rats ( x̅ ± s)

| | | Hyp (μg · g⁻¹ | | |
|---|---|---|---|---|
| Groups | N | liver weight) | ALT(U · ml⁻¹) | AST(U · ml⁻¹) |
| Normal | 6 | 178 ± 21 ** | 35.7 ± 3.7 ** | 64.2 ± 3.8 ** |
| Model | 10 | 524 ± 524 | 258.8 ± 27.1 | 285.6 ± 29.5 |
| Cordyceps sinensis | 10 | 371 ± 50 ** # | 177.5 ± 47.0 ** # | 209.3 ± 40.7 ** # |
| polysaccharide | | | | |
| Gypenosides | 11 | 390 ± 71 * # | 216.5 ± 51.9 * ## | 243.3 ± 49.6 * ## |
| Amygdaloside | 10 | 403 ± 89 * # | 220.8 ± 47.4 * ## | 249.1 ± 47.4 ## |
| Composition A | 11 | 293 ± 99 ** | 136.2 ± 44.9 ** | 164.6 ± 44.8 ** |
| Fuzhenghuayu | 11 | 283 ± 48 ** | 134.8 ± 42.4 ** | 163.2 ± 42.3 ** |
| capsule | | | | |
| Colchicine | 11 | 373 ± 112 * | 192.6 ± 60.9 ** # | 220.9 ± 60.9 ** # |

TABLE 11
Activity of GGT and content of Alb and Tbil of blood serum of rats ( x̅ ± s)

| Groups | N | GGT(U · ml⁻¹) | Alb(g · l⁻¹) | Tbil (mg · dl⁻¹) |
|---|---|---|---|---|
| Normal | 6 | 5.34 ± 0.96 ** | 31.93 ± 1.12 ** | 0.51 ± 0.12 ** |
| Model | 10 | 20.80 ± 8.83 | 22.03 ± 3.16 | 1.02 ± 0.25 |
| Cordyceps sinensis | 10 | 10.62 ± 3.16 ** # | 25.13 ± 2.41 * # | 0.79 ± 0.23 * # |
| polysaccharide | | | | |
| Gypenosides | 11 | 11.37 ± 4.17 ** # | 25.31 ± 2.62 * # | 0.70 ± 0.18 ** |
| Amygdaloside | 10 | 16.10 ± 6.74 ## | 23.67 ± 2.84 ## | 0.85 ± 0.23 # |
| Composition A | 11 | 7.95 ± 2.55 ** | 27.18 ± 2.03 ** | 0.60 ± 0.18 ** |
| Fuzhenghuayu | 11 | 8.40 ± 2.50 ** | 26.11 ± 3.30 ** | 0.61 ± 0.17 ** |
| capsule | | | | |
| Colchicine | 11 | 13.87 ± 9.13 | 24.59 ± 2.11 * ## | 0.74 ± 0.25 * |

Note:
Compared with the model group,
* p < 0.05,
** p < 0.01;
Compared with the composition A group,
# p < 0.05,
## p < 0.01.

Conclusion: the composition of the invention had significant therapeutical effect on rats having CCl₄-induced liver fibrosis and injury, and the effect was better than a single component.

While particular embodiments of the invention have been shown and described, it will be obvious to those skilled in the art that changes and modifications may be made without departing from the invention in its broader aspects, and therefore, the aim in the appended claims is to cover all such changes and modifications as fall within the true spirit and scope of the invention.

The invention claimed is:

1. A pharmaceutical composition for treatment of chronic liver diseases, comprising cordyceps sinensis polysaccharide, amygdaloside, and gypenoside.

2. The pharmaceutical composition of claim 1, wherein a weight ratio of said cordyceps sinensis polysaccharide to said amygdaloside to said gypenoside is 1:0.2-10:0.2-8.

3. The pharmaceutical composition of claim 2, wherein a weight ratio of said cordyceps sinensis polysaccharide to said amygdaloside to said gypenoside is 1:8:2.5.

4. The pharmaceutical composition of claim 2, wherein a weight ratio of said cordyceps sinensis polysaccharide to said amygdaloside to said gypenoside is 1:1.33:0.83.

5. A method of treatment of chronic liver diseases comprising administering to a patient in need thereof a pharmaceutical composition comprising cordyceps sinensis polysaccharide, amygdaloside, and gypenoside.

6. The method of claim 5, wherein said chronic liver disease is chronic hepatitis.

7. The method of claim 5, wherein said chronic liver disease is liver fibrosis.

8. The method of claim 5, wherein said chronic liver disease is liver cirrhosis.
